(19) United States
(12) Patent Application Publication
(10) Pub. No.: US 2007/0286902 A1
(43) Pub. Date: Dec. 13, 2007

(54) Dosage forms comprising a short acting sedative-hypnotic or salt thereof

(75) Inventors: Jianbo Xie, Davie, FL (US); Timothy Weng, Cooper City, FL (US); Mongkol Sriwongjanya, Pembroke Pines, FL (US)

Correspondence Address: DAVIDSON, DAVIDSON & KAPPEL, LLC, 485 SEVENTH AVENUE, 14TH FLOOR, NEW YORK, NY, 10018, US

(73) Assignee: ABRIKA Pharmaceuticals, Sunrise, FL (US)

(21) Appl. No.: 11/448,199

(22) Filed: Jun. 7, 2006

(51) Int. Cl. A61K 31/4745; A61K 9/22

(52) U.S. Cl. 424/468; 514/303

(57) ABSTRACT

In certain embodiments the invention is directed to an oral solid pharmaceutical dosage form comprising: a first portion of a short acting sedative-hypnotic in an release immediate release component; and a second portion of the sedative-hypnotic in a delayed component, the delayed release component comprising (i) a unitary core comprising the second portion of sedative-hypnotic dispersed in a controlled release matrix and (ii) a delayed release coating surrounding the core.

The present invention relates to dosage forms comprising a short acting sedative-hypnotic or pharmaceutically acceptable salt thereof.

### BACKGROUND OF THE INVENTION

Short acting sedative-hypnotic include compounds such as pyrazolopyrimidines (e.g., zaleplon); cyclopyrrolones, (e.g., zopiclone and its enantiomers); benzodiazepines (e.g., triazolam, temazepam and brotizolam); phenothiazines (e.g.; alimemazine); and imidazopyridines (e.g., zolpidem).

Zolpidem tartrate is indicated for the short-term treatment of insomnia and is marketed as Ambien CR® by Sanofi-Synthelabo Inc., in 6.25 mg and 12.5 mg tablets.

Ambien CR® tablets consist of a coated two-layer tablet: one layer that releases its drug content immediately and another layer that allows a slower release of additional drug content. The 6.25-mg Ambien CR tablet contains the following inactive ingredients: colloidal silicon dioxide, hypromellose, lactose monohydrate, magnesium stearate, microcrystalline cellulose, polyethylene glycol, potassium bitartrate, red ferric oxide, sodium starch glycolate, and titanium dioxide. The 12.5-mg Ambien CR tablet contains the following inactive ingredients: colloidal silicon dioxide, FD&C Blue #2, hypromellose, lactose monohydrate, magnesium stearate, microcrystalline cellulose, polyethylene glycol, potassium bitartrate, sodium starch glycolate, and titanium dioxide, and yellow ferric oxide.

The FDA publication entitled “Approved Drug Products with Therapeutic Equivalence”, commonly referred to as the “Orange Book” lists U.S. Pat. Nos. 4,382,938 and 6,514,531 as purportedly encompassing the active ingredient of Ambien CR® tablets (i.e., zolpidem tartrate).

Chemically, zolpidem tartrate is N,N,6-trimethyl-2-p-tolylimidazo[1,2-a]pyridine-3-acetemide L-(+)-tartrate (2:1). Chemically urrelated to other drugs with known hypnotic properties such as the benzodiazepines and barbiturates, zolpidem interacts with a GABA-BZ receptor complex and shares some of the pharmacological properties of benzodiazepines. Zolpidem's pharmacological action is via subunit modulation of the GABA_{A} receptor chloride channel macromolecular complex. The major modulatory site of the GABA_{A} receptor complex is located on its alpha (α) subunit and is referred to as the benzodiazepine (BZ) or omega (ω₁) receptor. In contrast to the benzodiazepines, which nonselectively bind to and activate all omega receptor subtypes, zolpidem has been reported to preferentially bind the (ω₁) receptor with a high affinity ratio of the alpha₁/alpha₅ subunits.

Zolpidem is characterized by rapid absorption from the GI tract and a short elimination half-life (t_{1/2}). Zolpidem is converted to inactive metabolites, which reduces the possibility of residual next-day effects from prolonged or excessive sedation. Pharmacokinetic and pharmacodynamic data show that zolpidem has both a rapid absorption and onset of hypnotic action. Following oral administration, zolpidem demonstrates linear kinetics in the therapeutic dosage range, which is typically about 5 to about 20 mg. CNS depression with impairment of cognitive and motor function, commonly seen with barbiturates or long-acting benzodiazepines in the treatment for insomnia, is not common with zolpidem.

Controlled release short acting sedative-hypnotic formulations have been described in U.S. Pat. Nos. 6,514,531; 6,638,535; and 6,485,746 and in U.S. Publication Nos. 2004/0258750 A1; 2003/0054042 and 2003/0091632 A1.

There continues to exist a need in the art for a controlled release dosage form of a short acting sedative-hypnotic (e.g., zolpidem) for the treatment of insomnia.

### OBJECTS AND SUMMARY OF INVENTION

It is an object of the invention to provide a controlled release dosage form of a short acting sedative-hypnotic, such as zolpidem tartrate.

It is an object of certain embodiments of the invention to provide a method of preparing a controlled release dosage form of a short acting sedative-hypnotic as disclosed herein.

It is an object of further embodiments of the invention to provide a method of treating insomnia comprising administering to a patient in need thereof, a controlled release dosage form of a short acting sedative-hypnotic, as disclosed herein.

It is an object of other embodiments of the invention to provide a controlled release dosage form comprising a short acting sedative-hypnotic which releases the active agent over a predetermined time period compatible with the desired time of sleep, and the time needed for elimination of the drug from the human body to a sufficiently low level.

It is an object of certain embodiments of the invention to provide a controlled release dosage form comprising a short acting sedative-hypnotic which provides sleep for a sufficient time, e.g., about 6 to about 9 hours, or from about 7 to about 8 hours and which does not result in significant next-day residual effects (also referred to as “hangover” effect).

It is a further object of the invention to provide a controlled release dosage form which provides a hypnotic effect throughout the night and does not disturb the alternating stages of REM and non-REM sleep, thus eliminating the need to awaken during sleep to administer an additional dose.

It is another object of the invention to provide a controlled release dosage form which promotes falling asleep by virtue of rapid onset of action.

It is a further object of certain embodiments of the invention to provide a controlled release dosage form which promotes staying asleep by virtue of controlled release. Further objects of certain embodiments of the invention include providing a better night's sleep and awaking feeling rested and refreshed as opposed to feeling groggy.

In accordance with the above objects, the present invention is directed, in part, to a controlled release oral pharmaceutical dosage form comprising a therapeutically effective amount of a short-acting sedative-hypnotic, the formulation providing an in-vitro dissolution wherein not less than about 70% or not less than about 75% of the short-acting sedative-hypnotic is released within 30 minutes, utilizing USP Apparatus II paddle method at 50 rpm in 0.01N HCL solution.

In certain embodiments, the oral pharmaceutical dosage form in accordance with the present invention comprises a first portion of a short acting sedative-hypnotic in a controlled release component and a second portion of the short-acting sedative-hypnotic in an immediate release component. In further preferred embodiments, the present invention is directed to an oral solid pharmaceutical dosage form comprising a first portion of a short acting sedative-hypnotic in an immediate release component; and a second portion of the sedative-hypnotic in a controlled release component, the delayed release component comprising (i) a unitary core comprising the second portion of sedative-hypnotic dispersed in a controlled release matrix and (ii) a delayed release coating surrounding the unitary core.

In other embodiments, the present invention is directed to a method of preparing an oral pharmaceutical dosage form comprising a) dispersing a first portion of a short acting sedative-hypnotic in a controlled release matrix to form a unitary core; b) coating the unitary core with an effective amount of a delayed release coating; and c) overcoating the delayed release coated unitary core with a second portion of the sedative-hypnotic. In certain preferred embodiments, the unitary core of the formulation disclosed herein is a compressed tablet. In further preferred embodiments, the second portion of the sedative hypnotic is in immediate release form and is coated over the delayed release coating.

In further embodiments, formulations in accordance with the invention release from about 70% to about 90% of the hypnotic at 0.5 hour; from about 80% to about 100% at 2 hours and greater than 90% at 4 hours, when subjected to in-vitro dissolution utilizing USP Apparatus I paddle method at 50 rpm, in a pH 6.8 buffer solution.

In other aspects, formulations in accordance with the invention release not less than about 70% or not less than about 75% of the short-acting sedative-hypnotics is released within 30 minutes, utilizing USP Apparatus II paddle method at 50 rpm in 0.01N HCL solution; and from about 70% to about 90% of the hypnotic at 0.5 hour; from about 80% to about 100% at 2 hours and greater than 90% at 4 hours, when subjected to in-vitro dissolution utilizing USP Apparatus I paddle method at 50 rpm, in a pH 6.8 buffer solution.

The controlled release matrix of the formulations in accordance with the invention may comprise a controlled release material selected from the group consisting of alkylcelluloses, acrylic and methacrylic acid polymers and copolymers, cellulose ethers, hydroxyalkylcelluloses, carboxyalkylcelluloses, waxes, gums, and combination thereof.

The controlled release matrix of the formulation disclosed herein may also comprise an organic acid, e.g., maleic, tartaric, malic, fumaric, lactic, citric, adipic and/or succinic acid.

In further embodiments, the controlled release matrix of the formulation in accordance with the invention comprises from about 20% to about 80% by weight of a controlled release material; from about 10% to about 40% by weight of microcrystalline cellulose; from about 10% to about 60% by weight of a pharmaceutically acceptable inert diluent; from about 1% to about 10% by weight of an organic acid; from about 0.1% to about 5% by weight of fumed silica and from about 0.1 to about 2% by weight of a pharmaceutically acceptable lubricant.

In preferred embodiments, the controlled release matrix core of the dosage form of the present invention comprises from about 20% to about 40% by weight of the dosage form of a controlled release material; from about 10 to about 30% by weight of the dosage form of silicified microcrystalline cellulose; from about 25 to about 35% by weight of the dosage form of a pharmaceutically acceptable inert diluent; from about 5 to about 10% by weight of the dosage form of an organic acid; from about 0.1 to about 1% by weight of the dosage form of fumed silica and from about 1 to about 2% by weight of the dosage form of a pharmaceutically acceptable lubricant.

In preferred embodiments, the sedative-hypnotic is homogenously dispersed in the controlled release matrix. In further preferred embodiments, the sedative-hypnotic is zolpidem or a pharmaceutically acceptable salt thereof. (e.g. zolpidem tartrate).

The invention is also directed to a method of inducing sleep in a human patient comprising administering to a patient in need thereof a formulation as contemplated herein.

By “homogeneous” it is meant for purposes of the present invention that the active agent is dispersed uniformly or substantially uniformly throughout the controlled release matrix.

The term “short acting sedative-hypnotic” means a compound administered in the treatment of insomnia which has an elimination half-life sufficiently small as to reduce or eliminate a “hangover” effect in the patient upon wakening in the morning after sleep. Generally, the t ½ of the compound should be less than about 3 hours.

### BRIEF DESCRIPTION OF THE DRAWINGS

FIG. 1 shows the dissolution profile for the formulation of Example 1, tested using the USP Apparatus II paddle method at 50 rpm in 0.01N HCL.

FIG. 2 shows the dissolution profile for the formulation of Example 1, tested using the USP Apparatus I basket method at 50 rpm in a pH 6.8 buffer solution.

FIG. 3 shows the dissolution profile for the formulation of Example 2, tested using the USP Apparatus II paddle method at 50 rpm in 0.01N HCL medium.

FIG. 4 shows the dissolution profile for the formulation of Example 2, tested using the USP Apparatus I basket method at 50 rpm in a pH 6.8 buffer solution.

### DETAILED DESCRIPTION OF THE INVENTION

The present invention is directed to a controlled release pharmaceutical dosage form comprising a sedative hypnotic, preferably zolpidem tartrate. The formulation may include a first portion of the active agent, in order to induce sleep without the necessity to have an extended “lag time” for drug absorption. The formulation may also include a second portion of the active agent in a controlled release component, in order to maintain sleep throughout the night without the need to administer a second dose.

Preferably, the dosage form releases not less than about 70% or not less than about 75% of the short-acting sedative-hypnotic within 30 minutes, utilizing USP Apparatus II paddle method at 50 rpm in 0.01N HCL solution; and from about 70% to about 90% of the hypnotic at 0.5 hour; from about 80% to about 100% at 2 hours and greater than 90% at 4 hours, when subjected to in-vitro dissolution utilizing USP Apparatus I paddle method at 50 rpm, in a pH 6.8 buffer solution.

The controlled release dosage forms of the present invention preferably provide effective blood levels of a short acting sedative-hypnotic or pharmaceutically acceptable salt thereof for a suitable time, e.g., about 8 hours, to maintain sleep in the treatment of insomnia.

“Pharmaceutically acceptable salts” of a sedative-hypnotic, as used herein, is meant to encompass all pharmaceutically acceptable salts, including, but not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; inorganic acid salts such as hydrochloride, hydrobromide, sulfate, phosphate and the like; organic acid salts such as formate, acetate, trifluoroacetate, maleate, fumarate, tartrate and the like; sulfonates such as methanesulfonate, benzenesulfonate, p-toluenesulfonate, and the like; amino acid salts such as arginate, asparginate, glutamate and the like. The preferred salt form for use in accordance with the present invention is the tartrate salt.

Suitable short acting sedative-hypnotics useful in the present invention include compounds (including their salt forms) such as pyrazolopyrimidines (e.g., zaleplon); cyclopyrrolones, (e.g., zopiclone and its enantiomers); benzodiazepines (e.g., triazolam, temazepam and brotizolam); phenothiazines (e.g.; alimemazine); and imidazopyridines (e.g., zolpidem). The preferred short acting sedative-hypnotic for use in the present invention is zolpidem tartrate.

When the formulation of the present invention include zolpidem or a pharmaceutically acceptable salt thereof, the active agent can be included in an amount, e.g., from about 1 mg to about 25 mg, or from about 5 to 20 mg.

In certain embodiments, the dosage form of the present invention comprises a first portion of a short acting sedative-hypnotic in an immediate release component; and a second portion of the sedative-hypnotic in a controlled release component, the controlled release component comprising (i) a unitary core comprising the second portion of sedative-hypnotic dispersed in a controlled release matrix and (ii) a delayed release coating surrounding the unitary core.

The immediate release portion allows for the short acting sedative-hypnotic to be immediately released, thus inducing a quick onset of sleep. Further release of the sedative-hypnotic is delayed by virtue of the delayed release coating layer. Once the delayed release coating is dissolved, the remainder of the dosage form is released at a controlled rate by virtue of the controlled release matrix. The controlled release of the sedative-hypnotic preferably provides a hypnotic effect throughout the night without the need to awaken to administer an additional dose.

A non-limiting list of suitable controlled-release materials which may be included in the matrix core according to the invention include hydrophilic and/or hydrophobic materials such as polymers, protein derived materials, waxes, shellac, gums, hydrogels, and oils such as hydrogenated castor oil and hydrogenated vegetable oil. Suitable polymers include alkylcelluloses (such as ethylcellulose), acrylic and methacrylic acid polymers and copolymers (such as Eudragit® commercially available by Rohm Pharma), alkylvinyl polymers, cellulose ethers, (such as hydroxyalkylcelluloses e.g., hydroxypropylmethylcellulose) and carboxyalkylcelluloses. Examples of acrylic and methacrylic acid polymers and copolymers include methyl methacrylate, methyl methacrylate copolymers, ethoxyethyl methacrylates, ethyl acrylate, trimethyl ammonioethyl methacrylate, cyanoethyl methacrylate, aminoalkyl methacrylate copolymer, poly(acrylic acid), poly(methacrylic acid), methacrylic acid alkylamine copolymer, poly(methyl methacrylate), poly(methacrylic acid)(anhydride), polymethacrylate, polyacrylamide, poly(methacrylic acid anhydride), and glycidyl methacrylate copolymers. Waxes include, for example, natural and synthetic waxes, fatty acids, fatty alcohols, and mixtures of the same (e.g., beeswax, carnauba wax, stearic acid and stearyl alcohol). Certain embodiments of the present invention utilize mixtures of any of the foregoing controlled release materials in the matrix core. However, any pharmaceutically acceptable hydrophobic or hydrophilic controlled-release material which is capable of imparting controlled-release of the active agent may be used in accordance with the present invention.

Cellulosic polymers which may be used in the core of the present invention include hydroxyethylcellulose, hydroxypropylmethylcellulose, hydroxypropylcellulose, sodium carboxymethylcellulose, and mixtures thereof. A preferred controlled release carrier is hydroxypropylmethylcellulose (“HPMC”). HPMC polymers are available from Dow Chemical under the trade name METHOCEL®.

In certain embodiments, the controlled release material further comprises effective amounts of different grades of hydroxypropylmethylcellulose (HPMC), commercially available as Methocel K4M® and Methocel E5® by The Dow Chemical Company (Midland, Mich.).

Another example of a class of polymers that may be used in the present invention is carbomers. Carbomers are synthetic high-molecular-weight polymers of acrylic acid that are cross-linked with either allylsucrose or allyl ethers of pentaerythritol. Carbomers are typically used as dry or wet binders and as a rate controlling excipient. Certain carbomers for use in certain embodiments of the present invention include for example, Carbopol® 941, 971 PNF, 981 and 71G manufactured by Noveon, Inc.

In addition to the above ingredients, in certain embodiments the controlled release matrix core of the present invention may further include a wide variety of additives and excipients that enhance drug solubility or, that promote stability, tableting or processing. Such additives and excipients include tableting aids, lubricants, surfactants, fillers or diluents, water-soluble polymers, pH modifiers, binders, pigments, disintegrants, glidants, plasticizer, solvents, flow conditioning agents, suspending agents, viscosity-increasing agents, anti-caking agents, antioxidants, lubricants and flavorants. Examples of such components are metallic salts of acids such as aluminum stearate, calcium stearate, magnesium stearate, sodium stearate, and zinc stearate; fumed or colloidal silica which is commercially available as Cab-O-Sil M5®, by Cabot Corporation; povidone, fatty acids, hydrocarbons and fatty alcohols such as stearic acid, palmitic acid, liquid paraffin, stearyl alcohol, and palmitol; fatty acid esters such as glyceryl (mono- and di-) stearates, triglycerides, glyceryl (palmiticstearic) ester, sorbitan monostearate, saccharose monostearate, saccharose monopalmitate, and sodium stearyl fumarate; alkyl sulfates such as sodium lauryl sulfate and magnesium lauryl sulfate; polymers such as polyethylene glycols, polyoxethylene glycols, and polytetrafluoroethylene; and inorganic materials such as talc and dicalcium phosphate; sugars such as lactose, xylitol, sucrose, dextrose, fructose, sorbitol, mannitol, starches, other polyols, mixtures thereof and the like; and sodium starch glycolate. The quantities of these additional materials will be sufficient to provide the desired effect to the desired formulation. Specific examples of pharmaceutically acceptable carriers and excipients that may be used to formulate oral dosage forms are described in the Handbook of Pharmaceutical Excipients, American Pharmaceutical Association (1986), incorporated by reference herein.

Examples of lubricants include stearic acid, magnesium stearate, carnauba wax, glyceryl behenate, talc, mineral oil (in polyethylene glycol), mixtures thereof, and the like. Magnesium stearate and carnauba wax are preferred lubricants.

Examples of binders include water-soluble polymers, such as modified starch, gelatin, polyvinylpyrrolidone, polyvinyl alcohol, povidone, sodium carboxymethylcellulose, alginic acid, polyethylene glycol, polypropylene glycol, guar gum, polysaccharides, bentonite clay, sugar, poloxamer, collagen, albumin, gelatin, mixtures thereof, and the like.

Examples of fillers or diluents for use in the present invention include lactose, microcrystalline cellulose, dextrin, dextrose, starch, mixtures thereof and the like.

Examples of glidants for use in the present invention include calcium phosphate tribasic, calcium silicate, powdered cellulose, colloidal silicon dioxide, magnesium silicate, magnesium trisilicate, silicon dioxide, starch, talc, mixtures thereof and the like.

Direct compression vehicles may be used in the present invention and include, for example, processed forms of cellulose, sugars, and dicalcium phosphate dihydrate, among others. Microcrystalline cellulose is an example of a processed cellulose that is suitable as a direct compression vehicle for solid dosage forms.

Silicified microcrystalline cellulose is a particularly useful direct compression vehicle. Silicified microcrystalline cellulose is a particulate agglomerate of coprocessed microcrystalline cellulose and from about 0.1% to about 20% silicon dioxide, by weight of the microcrystalline cellulose, the microcrystalline cellulose and silicon dioxide being in intimate association with each other, and the silicon dioxide portion of the agglomerate being derived from a silicon dioxide having a particle size from about 1 nanometer (nm) to about 100 microns (μm), based on average primary particle size. Preferably, the silicon dioxide comprises from about 0.5% to about 10% of the silicified microcrystalline cellulose, and most preferably from about 1.25% to about 5% by weight relative to the microcrystalline cellulose. Moreover, the silicon dioxide preferably has a particle size from about 5 nm to about 40 μm, and most preferably from about 5 nm to about 50 μm. Moreover, the silicon dioxide preferably has a surface area from about 10 m² g to about 500 m²/g, preferably from about 50 m²/g to about 500 m²/g, and more preferably from about 175 m²/g to about 350 m²/g.

In certain embodiments of the present invention, the controlled release matrix core may further include an effective amount of a pharmaceutically acceptable organic acid. The pharmaceutically acceptable organic acid can be chosen, for example, among maleic, tartaric, malic, fumaric, lactic, citric, adipic or succinic acid and their acid salts where these exist, in the form of racemates or isomers, where these exist.

In preferred aspects of the invention, the organic acid is tartaric acid, and its acid salts.

In certain aspects of the present invention, the controlled release matrix core is coated with a delayed release coating, e.g., an enteric coating. Examples of suitable enteric polymers to be used for the enteric coating include cellulose acetate phthalate, hydroxypropyl-methylcellulose phthalate, polyvinylacetate phthalate, methacrylic acid copolymer, shellac, hydroxypropylmethylcellulose succinate, cellulose acetate trimellitate, and mixtures of any of the foregoing. An example of a suitable commercially available enteric material is available under the trade name Eudragit® L30D55 or Acryl-Eze®.

The enteric coating may be applied in any pharmaceutically acceptable manner known to those skilled in the art. For example, in one embodiment, the coating is applied via a fluidized bed. In another embodiment, the coating is applied via a coating pan. In certain embodiments, the enteric coating further includes a binder. Examples of suitable binders for use in the present invention are listed above.

In certain embodiments, the dosage form of the present invention further includes an immediate release portion. In one embodiment, the immediate release portion is over the delayed release coating disclosed above (with or without an intermediate layer, such as a film coat). The immediate release portion of the dosage form includes a portion of the short acting sedative-hypnotic. In another embodiment, the immediate release component can be separate and distinct from the enteric coated matrix, e.g., in the form of mutliparticulate, a tablet, or a powder. This separate and distinct component can be included with the enteric coated matrix in a capsule.

In certain embodiments, the immediate release portion of the dosage form of the present invention further includes a film coat that rapidly disintegrates or dissolves in water or the environment of use. The film coat may be a conventional sugar or polymeric film coating which is applied in a coating pan or by conventional spraying techniques. Preferred materials for the film coat are hydroxypropylmethylcellulose, polyvinyl alcohol, or mixtures thereof. An example of a commercially available film coat is under the Opadry tradename (e.g., Opadry® II, Yellow), from Colorcon, West Point, Pa.

### DETAILED DESCRIPTION OF THE PREFERRED EMBODIMENTS

The following examples illustrate various aspects of the present invention. They are not to be construed to limit the claims in any manner whatsoever.

### EXAMPLE 1

In Example 1, zolpidem tartrate 6.25 mg delayed release tablets including a delayed release portion (A) and an immediate release portion (B), were prepared in accordance with the present invention in three steps as follows:

The ingredients of the controlled release matrix core of the formulation of Example 1 are set forth in Table 1 below:

TABLE 1

| Ingredients | Percent (%) | Wt (mg)/Tablet |
|---|---|---|
| Zolpidem | 1.00 | 1.25 |
| Tartrate | | |
| Hydroxypropyl- | 5.00 | 6.25 |
| methylcellulose | | |
| (Methocel, K4M ®) | | |
| Hydroxypropyl- | 30.00 | 37.50 |
| methylcellulose | | |
| (Methocel E5 ®) | | |
| Silicified Microcrystalline | 20.00 | 25.00 |
| Cellulose | | |
| (Prosolv, HD90 ®) | | |
| Lactose, | 34.35 | 42.94 |
| Anhydrous | | |
| Tartaric Acid Powder | 8.40 | 10.50 |
| (pass #40 mesh) | | |
| Colloidal silica | 0.50 | 0.62 |
| (Cab-O-Sil, M-5 ®) | | |
| Magnesium Stearate | 0.75 | 0.94 |
| TOTAL | 100 | 125.0 |

The controlled release matrix core of the formulation of Example 1 was prepared as follows:
1. All the ingredients listed in Table 1 above were weighed.
2. The Tartaric Acid was passed through a CoMil.
3. Methocel K4M®, Tartaric acid, Zolpidem Tartrate, ProSolv®, Cab-O-Sil®, and Lactose Anhydrous were charged into a V-blender and blended for 5 minutes.
4. The blend was discharged and passed through a CoMil®.
5. Methocel E5® were passed through the CoMil®.
6. The material from Step#3, Step#4 and Step#5 were blended in a Blender for 10 minutes.
7. Magnesium Stearate was screened through a #25 mesh screen.
8. The screened Magnesium Stearate was added to the blender and mix for 5 minutes.
9. The blend was compressed with weights of 125 mg using the 7 mm standard concave punch.

The ingredients of the enteric coated formulation of Example 1 are listed in Table 2 below:

TABLE 2

| Ingredients | Percent (%) | Mg/tablet |
|---|---|---|
| Zolpidem core tablets | 94.50 | 125 |
| 1.25 mg | | |
| Hydroxypropyl- | 1.00 | 1.32 |
| methylcellulose | | |
| (Methocel E5 ®) | | |
| Water* | | |
| Methacrylic acid | 4.05 | 5.36 |
| copolymer | | |
| (Acry-EZE ®) | | |
| Povidone | 0.45 | 0.60 |
| (Killion k 30) | | |
| Water* | | |
| TOTAL | 100.0 | 132.3 |

*Will be evaporated during the process.

The enteric coated formulation of Example 1 was prepared as follows:
1. All the ingredients of Table 2 were weighed.
2. Methocel® E5 was dissolved into water and stirred.
3. Separately, Povidone (killion k-30) was added into water and stirred until dissolved.
4. The Acryl-EZE® was dispersed into the above solution above and stirred for at least 30 minutes before use.
5. The tablets were charged into the perforated coating pan. The tablets were warmed to reach 40±4 C.
6. The spraying of the dispersion from step#4 was started and the recording of the in-process data begun.
7. The tablets were dried for 10 minutes after completion.
8. The Methocel® E5 solution from Step #2 was sprayed onto the tablets.
9. When the weight gain reached 4.5%, the spraying stopped the tablets were dried for 10 minutes.

The ingredients of the final formulation of Example 1, including the immediate release portion, are listed in Table 3 below:

TABLE 3

| | | Wt (mg)/ |
|---|---|---|
| Ingredients | Percent (%) | Tablet |
| Zolpidem Enteric Coated | 92.68 | 132.3 |
| Tablets, 1.25 mg | | |
| Methocel E5 | 1.75 | 2.50 |
| Polytheylene glycol 400 | 0.44 | 0.63 |
| (PEG 400) | | |
| Zolpidem Tartrate | 3.50 | 4.99 |
| Opadry ® II, White | 1.63 | 2.33 |
| Water* | * | |
| Hydrochloride acid, USP | q.s. | q.s. |
| TOTAL | 100 | 142.7 |

*Will be evaporated during process.

The final formulation of by Example 1, including the immediate release portion was prepared as follows:
1. All ingredients in Table 3 were weighed.
2. The Methocel® E5 was added to water and stirred until it dissolved.
3. Hydrochloride Acid was added to adjust the solution pH to about 1.0.
4. Add Zolpidem Tartrate into the solution from Step#3 and mixed until it completely dissolved.
5. Add the PEG 400 to the solution and mix not less than 20 minutes before use.
6. Separately, the Opadry II Yellow solution was prepared.
7. The enteric coated tablets were charged into a perforated coating pan.
8. The spraying of Zolpidem Tartrate solution from step #5 was started when the exhaust temperature reached 40±5 C and recording of the in-process data begun.
9. After the coating dispersion was fully exhausted, the tablets were dried for 10 minutes.
10. The Opadry II, White dispersion (from step#4) was sprayed under the same condition, recording of the parameters and the tablets dried for 10 minutes after spraying.

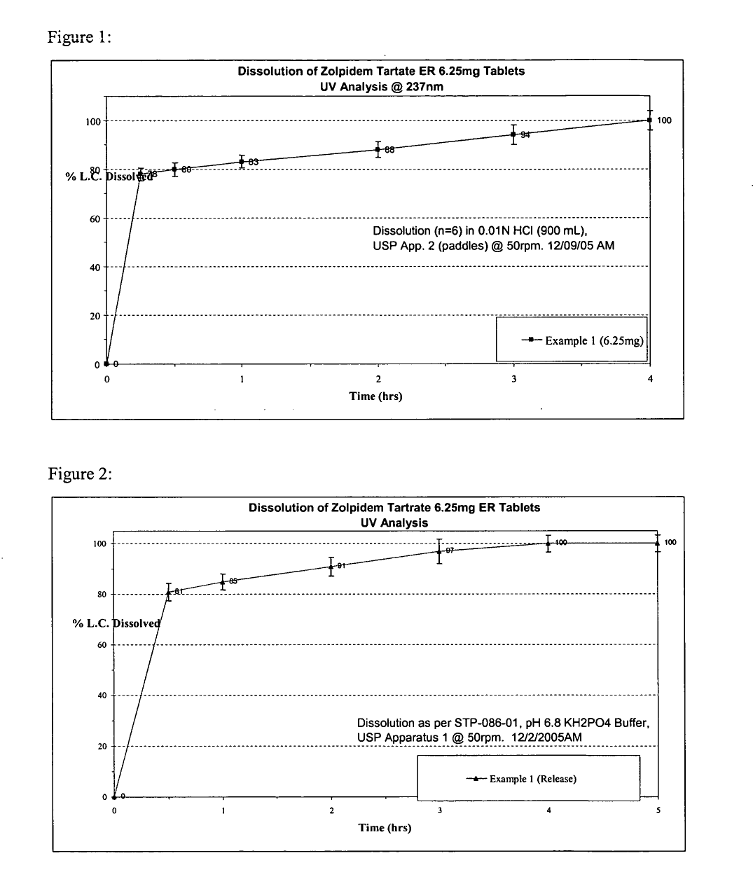

The tablets prepared in accordance with Example 1 were dissolution tested in USP dissolution Apparatus type II paddle method, in 0.01 NHCL with an agitation of 50 rpm. The dissolution results are illustrated in FIG. 1.

The tablets prepared in accordance with Example 1 were dissolution tested in USP dissolution Apparatus type I basket method, in a pH of 6.8 buffer solution with an agitation of 50 rpm. The dissolution results are illustrated in FIG. 2.

### EXAMPLE 2

In Example 2, zolpidem tartrate 12.5 mg delayed release tablets including a delayed release portion and an immediate release portion, were prepared in accordance with the present invention in three steps as follows:

The ingredients of the controlled-release matrix core of the formulation of Example 2 are listed in Table 4 below:

TABLE 4

| Ingredients | Percent (%) | Wt (mg)/Tablet |
|---|---|---|
| Zolpidem | 2.00 | 2.50 |
| Tartrate | | |
| Hydroxypropyl- | 5.00 | 6.25 |
| methylcellulose | | |
| (Methocel, K4M ®) | | |
| Hydroxypropyl- | 30.00 | 37.50 |
| methylcellulose | | |
| (Methocel E5 ®) | | |
| Silicified Microcrystalline | 20.00 | 25.00 |
| Cellulose | | |
| (Prosolv, HD90 ®) | | |
| Lactose, | 33.35 | 41.69 |
| Anhydrous | | |
| Tartaric Acid Powder | 8.40 | 10.50 |
| (pass #40 mesh) | | |
| Colloidal silica | 0.50 | 0.625 |
| (Cab-O-Sil, M-5 ®) | | |
| Magnesium Stearate | 0.75 | 0.94 |
| TOTAL | 100 | 125.0 |

The controlled-release matrix core of the formulation of Example 2 was prepared in accordance with the process of Example 1.

The ingredients of the enteric coated formulation of Example 2 are listed in Table 5 below:

TABLE 5

| | | Wt(g)/ |
|---|---|---|
| Ingredients | Percent (%) | Batch |
| Zolpidem core tablets | 94.50 | 125 |
| 2.5 mg | | |
| Hydroxypropyl- | 1.00 | 1.32 |
| methylcellulose | | |
| (Methocel ® E5) | | |
| Water* | | |
| Methacrylic acid | 4.05 | 5.36 |
| copolymer | | |
| (Acryl-EZE ®) | | |
| Povidone, USP | 0.45 | 0.60 |
| (Kollion k 30) | | |
| Water* | | |
| TOTAL | 100.0 | 132.3 |

*Will be evaporated during the process.

The enteric coated formulation of Example 2 was prepared in accordance with the process of Example 1.

The ingredients of the final formulation of Example 2, including the immediate release portion are listed in Table 6 below:

TABLE 6

| | | Wt (mg)/ |
|---|---|---|
| Ingredients | Percent (%) | Tablet |
| Zolpidem Enteric Coated | 87.70 | 132.3 |
| Tablets, 2.5 mg | | |
| Methocel ® E5 | 3.32 | 5.01 |
| Polytheylene glycol 400 | 0.83 | 1.25 |
| (PEG 400) | | |
| ZolpidemTartrate | 6.63 | 10.00 |
| Opadry II, Yellow | 1.52 | 2.29 |
| Water* | * | |
| Hydrochloride acid, USP | q.s. | q.s. |
| TOTAL | 100 | 150.9 |

*Will be evaporated during process.
** Opadry II is commercially available from Colorcon

The final formulation of Example 2, including the immediate release portion was prepared in accordance with the process of Example 1.

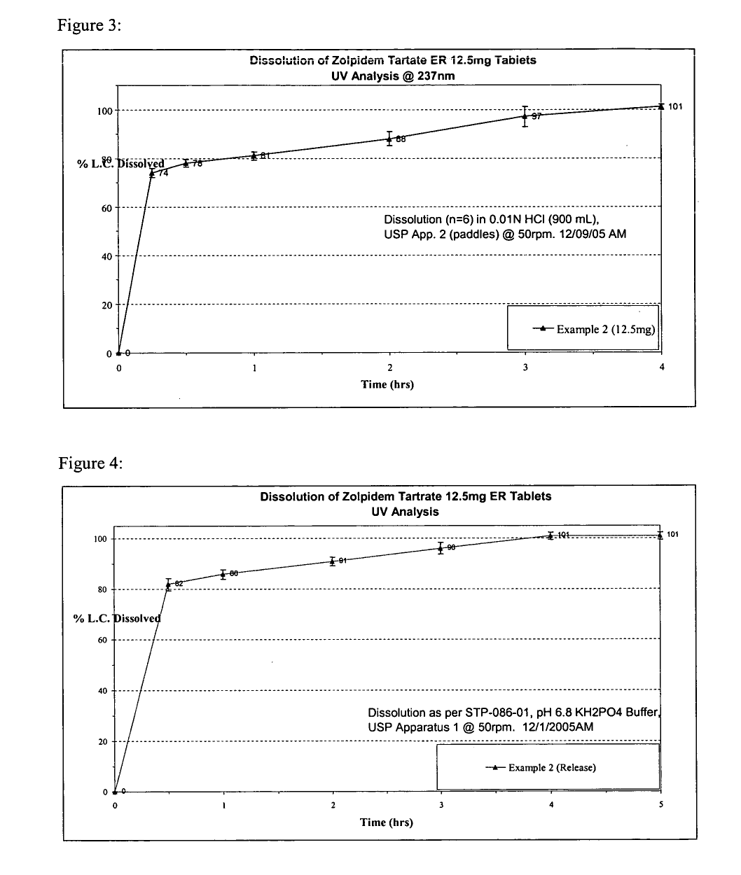

The tablets prepared in accordance with Example 2 were dissolution tested in USP dissolution Apparatus type II paddle method, in 0.01 NHCL with an agitation of 50 rpm. The dissolution results are illustrated in FIG. 3.

The tablets prepared in accordance with Example 2 were dissolution tested in USP dissolution Apparatus type I basket method, in a pH of 6.8 buffer solution with an agitation of 50 rpm. The dissolution results are illustrated in FIG. 4.

We claim:

1. An oral solid pharmaceutical dosage forms comprising:
a first portion of a short acting sedative-hypnotic in an immediate release component; and
a second portion of said sedative-hypnotic in a controlled release component, said controlled release component comprising (i) a unitary core comprising the second portion of sedative-hypnotic dispersed in a controlled release matrix and (ii) a delayed release coating surrounding said core.

2. An oral solid pharmaceutical dosage form comprising:
a first portion of a short acting sedative-hypnotic in an immediate release component; and
a second portion of said sedative-hypnotic in a controlled release component, the dosage form providing an in-vitro dissolution wherein not less than about 70% of said short-acting sedative-hypnotic is released after 0.5 hours, utilizing USP Apparatus II paddle method at 50 rpm in 0.01N HCL solution; and the
formulation releasing from about 80 to about 100% of the hypnotic at 2 hour; and
greater than 90% at 4 hours, when subjected to in-vitro dissolution utilizing USP Apparatus II paddle method at 50 rpm, in a pH 6.8 buffer solution.

3. A controlled release oral pharmaceutical dosage form comprising a therapeutically effective amount of a short acting sedative-hypnotic, said formulation providing an in-vitro dissolution wherein not less than about 70% of said short-acting sedative-hypnotic is released after 0.5 hours, utilizing USP Apparatus II paddle method at 50 rpm in 0.01N HCL solution; and the
formulation releasing from about 80 to about 100% of the hypnotic at 2 hour; and
greater than 90% at 4 hours, when subjected to in-vitro dissolution utilizing USP Apparatus II paddle method at 50 rpm, in a pH 6.8 buffer solution.

4. The dosage form of claim 1, wherein said core is a compressed tablet.

5. The dosage form of claim 1, wherein said immediate release portion is coated over the delayed release coating.

6. The dosage form of claim 5, comprising a layer between the immediate release portion and the delayed release coating.

7. The dosage form of claim 1, wherein said controlled release matrix comprises a controlled release material selected from the group consisting of alkylcelluloses, acrylic and methacrylic acid polymers and copolymers, cellulose ethers, hydroxyalkylcelluloses, carboxyalkylcelluloses, waxes, gums, polysachrides, povidone, copovidone and mixtures thereof.

8. The dosage form of claim 1, wherein said controlled release material is hydroxypropylmethylcellulose.

9. The dosage form of claim 1, wherein said controlled release matrix comprises an organic acid selected from the group consisting of maleic, tartaric, malic, fumaric, lactic, citric, adipic and succinic acid.

10. The pharmaceutical dosage form of claim 1, wherein said matrix comprises: from about 20% to about 80% by weight of a controlled release material; from about 10% to about 40% by weight of microcrystalline cellulose; from about 10% to about 60% by weight of a pharmaceutically acceptable inert diluent; from about 1% to about 10% by weight of an organic acid; from about 0.1% to about 5% by weight of fumed silica and from about 0.1% to about 2% by weight of a pharmaceutically acceptable lubricant.

11. The dosage form of claim 1, wherein said sedative-hypnotic is homogenously dispersed in said controlled release matrix.

12. The dosage form of claim 1, wherein said sedative-hypnotic is zolpidem or a pharmaceutically acceptable salt thereof.

13. The dosage form of claim 1, wherein said hypnotic is zolpidem tartrate.

14. The dosage form of claim 1, which releases not less than about 70% of said hypnotic at a 30 minutes, when subjected in-vitro dissolution utilizing USP Apparatus II paddle method at 50 rpm in 0.01N HCL solution.

15. The dosage form of claim 1, which releases not more than about 90% of said hypnotic at 30 minutes when subjected to in-vitro dissolution utilizing USP Apparatus II paddle method at 50 rpm in 0.01N HCL solution.

16. The dosage form of claim 1, which releases from about 70% to about 90% of said hypnotic at 0.5 hour; from about 80% to about 100% at 2 hours and greater than about 90% at 4 hours, when subjected to in-vitro dissolution utilizing USP Apparatus I basket method at 50 rpm, in a pH 6.8 buffer solution.

17. The pharmaceutical dosage form of claim 3, which comprises a first portion of a short acting sedative-hypnotic in a controlled release component and a second portion of said short acting sedative-hypnotic in an immediate release component.

18. A controlled release oral pharmaceutical dosage form according to claim 3, wherein said short acting sedative-hypnotic is zolpidem or a pharmaceutically acceptable salt thereof.

19. A controlled release oral pharmaceutical dosage form according to claim 3, wherein said short acting sedative-hypnotic is zolpidem tartrate.

20. A method of inducing sleep in a human patient comprising administering to a patient in need thereof a dosage form according to any of claims 1-19.

21. A method of preparing an oral dosage form comprising:
a) dispersing a first portion of a short acting sedative-hypnotic in a controlled release matrix to form a unitary core;
b) coating said core with a delayed release coating; and
c) coating the delayed release coated core with a second portion of the sedative-hypnotic.
